(19) United States
(12) Patent Application Publication
(10) Pub. No.: US 2005/0182495 A1
(43) Pub. Date: Aug. 18, 2005

(54) Prosthesis for aero-digestive fistulae

(76) Inventor: Rafael Carmelo Antonio Perrone, Buevos Aires (AR)

Correspondence Address: Stephan A. Pendorf;Pendorf & Cutliff, 5111 Memorial Highway, Tampa, FL, 33634-7356, US

(21) Appl. No.: 10/847,504

(22) Filed: May 17, 2004

(30) Foreign Application Priority Data
Feb. 13, 2004 (AR) P 04 01 00452

(51) Int. Cl. A61F 2/02

(52) U.S. Cl. 623/23.72

(57) ABSTRACT

A prosthesis for aero-digestive fistulae for the blocking of an aero-digestive fistulae in cases where the digestive tract does not present anfractuosities similar to those caused by tumors, being impossible to fix a stent in said tract, prosthesis that is of a soft material with elastic memory and comprehends a central part, its section being of approximately the same size of the fistula and two lateral bodies, of greater size, its medial faces being in contact with the perimeter of the fistular surfaces of both sides.

### BACKGROUND OF THE INVENTION

1. Field of the Invention

The present invention refers to prosthesis for aero-digestive fistulae, more particularly to a prosthesis for the blocking of an aero-digestive fistulae in cases where the digestive tract does not present anfractuosities similar to those caused by tumors being impossible to fix a stent in said tract.

2. Description of the Related Art

As already known, when a fistula as the abovementioned is produced, it is necessary to obstruct it by means of a prosthesis that prevents the passage of fluids towards the digestive tract and also from it.

In addition to the obstruction of the orifice, the prosthesis must remain very well fixed in the position it was collocated.

### SUMMARY OF THE INVENTION

With the prosthesis of the present invention a good obstruction, as well as a fixation of the same to the walls surrounding the fistula, is obtained.

The principle of fixation of the prosthesis is similar to the one achieved with the buttons of shirts, using the fistulous orifice as the eye.

The obstruction occurs not only due to the fact that the fistulous orifice is occupied with the central part of the prosthesis, but also to the medial faces of both bodies, that, having an ampler diameter than the central part, are in close contact with the perimeter of the fistular surface of both sides, that is to say, of the aerial tract and the digestive tract.

Being the prosthesis of a soft material with elastic memory, the bodies have a better bidirectional obstruction, as each one of them acts as a diaphragm that adjusts more as the pressure on the same is increased.

For an optimal result of the action of the prosthesis, the central part of said prosthesis must have a very precise size with respect to the diameter, length and shape, to the orifice and the direction of the fistula that must be obstructed.

Usually the bodies have the same diameter, in small fistulae, or of an ampler diameter, the one that is collocated in the digestive tract when the fistulae are bigger.

Although in the embodiment of the present invention the bodies are disc-shaped and flat, they can be round, oval or rectangular depending on the particular characteristics of each fistula and the compromised segment of the aerial tract.

The main object of the present invention is a prosthesis for aero-digestive fistulae, its novelty consisting in the fact that it is of a soft material with elastic memory and comprehends a central part, its section being approximately of the same size of the fistula and two lateral bodies, of greater size, its medial faces being in contact with the perimeter of the fistular surfaces of both sides.

In a preferred embodiment of the present invention said central part completely occupies the fistulous orifice.

In said preferred embodiment of the present invention the medial faces of the lateral bodies are flat.

In said preferred embodiment of the present invention said lateral bodies are disc-shaped.

In another preferred embodiment of the present invention said lateral bodies have different sizes.

The main object of the present invention as well as its advantages will be appreciated in the following description of a preferred embodiment of the same with reference to the drawings described hereinafter.

### BRIEF DESCRIPTION OF THE DRAWINGS

FIG. 1 is a view in perspective of the prosthesis according to the present invention;

FIG. 2 shows an anatomical section with the prosthesis collocated in a fistula;

FIGS. 3 and 4 are views of other embodiments of the prosthesis; and

FIG. 5 is a diagram of the relationship of the dimensions of the prosthesis.

### DETAILED DESCRIPTION OF THE INVENTION

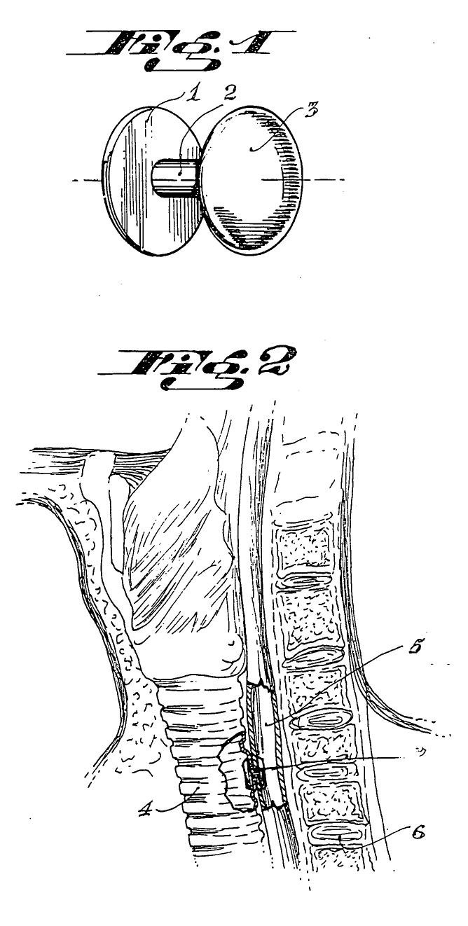

In FIG. 1 it is observed that the prosthesis comprehends two lateral bodies (1) and (3) joined by a central part (2) its size being smaller.

The material of the prosthesis is soft and has elastic memory so it can be deformed during its collocation and afterwards recover its definitive shape.

FIG. 2 shows a section in which the prosthesis is collocated in a fistula (7) produced between the trachea (4) and the esophagus (5), the section also showing the spine (6).

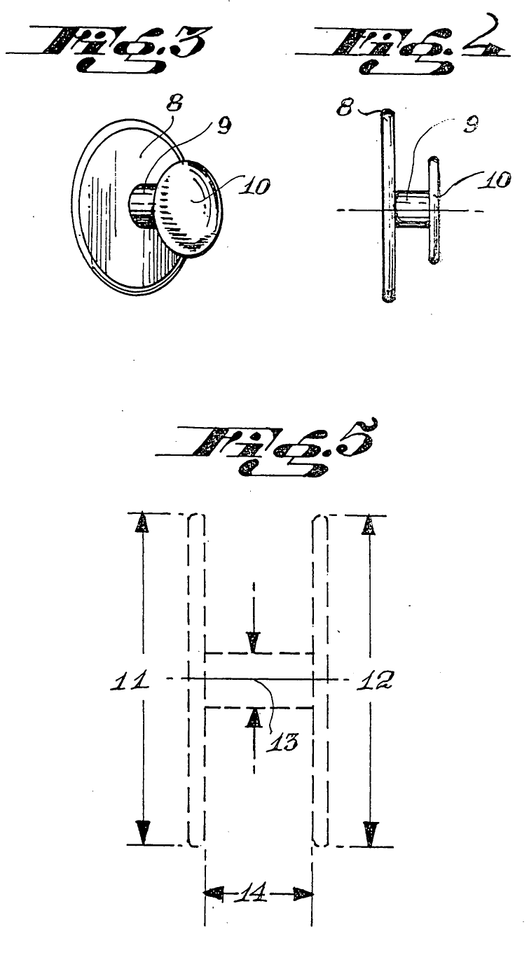

FIGS. 3 and 4 show another embodiment of the prosthesis in which the lateral body (8) is bigger in size than the lateral body (10), both bodies being joined by the central part (9).

In these figures one can observe that the medial faces of the bodies (8) and (10) are flat so the remain attached to the perimeter of the fistular surface.

FIG. 5 shows the prosthesis in an schematic manner indicating the measures to be taken in mind when said prosthesis has to be collocated in a fistula.

The measures (11) and (12) may be identical or not, depending of the size of the fistula, as in fistulae which are bigger, the size of the lateral body that is positioned in the digestive tract y bigger than the other lateral body.

The separation (14) between both lateral bodies must be equal to the sum of the width of the wall of the digestive tract and the width of the aerial tract, in order to achieve a close contact of the medial faces with the perimeter of the fistular surface.

The diameter (13) of the central part must be coincident with the size and shape of the fistula.

1. A prosthesis for aero-digestive fistulae wherein it is of a soft material with elastic memory and comprehends a central part, its section being of approximately the same size of the fistula and two lateral bodies, of greater size, its medial faces being in contact with the perimeter of the fistular surfaces of both sides.

2. The prosthesis for aero-digestive fistulae in accordance with claim 1, wherein said central part completely occupies the fistulous orifice.

3. The prosthesis for aero-digestive fistulae in accordance with claim 1, wherein the medial faces of the lateral bodies are flat.

4. The prosthesis for aero-digestive fistulae in accordance with claim 1, wherein said lateral bodies are disc-shaped.

5. The prosthesis for aero-digestive fistulae in accordance with claim 1, wherein said lateral bodies have different sizes.
